(19) United States
(12) Patent Application Publication
(10) Pub. No.: US 2023/0148936 A1
(43) Pub. Date: May 18, 2023

(54) ELECTROCARDIOGRAPHIC IMAGING USING PATCH ELECTRODES

(71) Applicant: CARDIOINSIGHT TECHNOLOGIES INC., INDEPENDENCE, OH (US)

(72) Inventors: Timothy G. LASKE, SHOREVIEW, MN (US); Kevin D. GOLLON, LAKEWOOD, OH (US); Qingguo ZENG, SOLON, OH (US); Qing LOU, SOLON, OH (US)

(21) Appl. No.: 17/936,676

(22) Filed: Sep. 29, 2022

Related U.S. Application Data
(60) Provisional application No. 63/278,634, filed on Nov. 12, 2021.

(51) Int. Cl. A61B 5/346; A61B 5/339; A61B 5/367; A61B 5/308; G16H 30/40

(52) U.S. Cl. CPC A61B 5/346; A61B 5/339; A61B 5/367; A61B 5/308; G16H 30/40; A61B 5/28

(57) ABSTRACT

The present disclosure provides a system that includes an arrangement of body surface electrodes on one or more patches adapted to be placed an outer surface of a patient's body. A computing apparatus includes non-transitory memory to store data and instructions executable by a processor thereof. The data includes anatomical geometry data, electrode geometry data and electrical data. The instructions can be programmed to register the anatomical geometry data and the electrode geometry data to provide co-registered geometry data representing the anatomy of the patient and the locations of the body surface electrodes in a common three-dimensional space. Electrophysiological signals can be reconstructed on a cardiac envelope of the heart based on the co-registered geometry data and the electrical data.

### CROSS-REFERENCE TO RELATED APPLICATION

This application claims the benefit of priority to U.S. provisional patent application No. 63/278,634, filed 12 Nov. 2021, which is incorporated herein by reference in its entirety.

### FIELD

The present technology is generally related to systems and methods of electrocardiographic imaging using patch-type electrodes.

### BACKGROUND

Electrocardiographic imaging (ECGI) is a non-invasive multi-lead ECG-type imaging tool that combines non-invasive electrical measurements with three-dimensional geometry of the heart and torso to reconstruct electrical signals onto the heart or another surface of interest. Mathematically, this is performed by solving the inverse problem. In one example, electrodes are implemented on a vest, and require medical imaging of the patient while wearing the vest prior to conducting an electrophysiology study. In addition to the relatively high cost of the vest, the preparation, collection and processing of preliminary data before the electrophysiology study tend to time consuming and require a high level of expertise.

### SUMMARY

The techniques of this disclosure generally relate to electrocardiographic imaging using patch-type electrodes.

In one aspect, the present disclosure provides a system that includes an arrangement of body surface electrodes on one or more patches adapted to be placed an outer surface of a patient's body. A computing apparatus includes non-transitory memory to store data and instructions executable by a processor thereof. The data includes anatomical geometry data, electrode geometry data and electrical data. The anatomical geometry data represents anatomy of the patient, which includes at least a portion of a heart and the outer surface of the patient's body, in three-dimensional space. The electrode geometry data represents locations of respective body surface electrodes in three-dimensional space. The electrode geometry data can be derived from at least one of (i) electrical signals measured by at least some of the electrodes, or (ii) a template describing the locations of respective body surface electrodes as applied to the patient. The electrical data represents electrophysiological signals measured by the electrodes. The instructions can be programmed to register the anatomical geometry data and the electrode geometry data to provide co-registered geometry data representing the anatomy of the patient and the locations of the body surface electrodes in a common three-dimensional space. Electrophysiological signals can be reconstructed on a cardiac envelope of the heart based on the co-registered geometry data and the electrical data.

In another aspect, the disclosure provides one or more non-transitory computer-readable media having instructions, which when executed by a processor, perform a method. The method can include emphasizing electrophysiological signals for a subset of measurement channels, corresponding to electrophysiological signals measured non-invasively by respective electrodes. The method can also include storing electrical data to represent the emphasized electrophysiological signals for the subset of measurement channels together with other electrophysiological signals for other measurement channels measured non-invasively by other electrodes. The method can also include reconstructing electrophysiological signals on a cardiac envelope of a heart based on co-registered geometry data and the electrical data including the emphasized electrophysiological signals. The co-registered geometry data represents the anatomy of the patient and spatial locations of the respective electrodes in a common three-dimensional space, and the electrical data represents electrophysiological signals measured non-invasively by electrodes corresponding to respective measurement channels.

In yet another aspect, the disclosure provides one or more non-transitory computer-readable media having instructions, which when executed by a processor, perform a method. The method includes selecting a proper subset of a plurality of measurement channels, the measurement channels corresponding to arrangement of electrodes on one or more patches adapted to measure electrophysiological signals on an outer surface of a patient's body. The method also includes storing electrical data to represent the electrophysiological signals measured for the selected subset of measurement channels. The method also includes reconstructing electrophysiological signals on a cardiac envelope of a heart based on co-registered geometry data and the electrical data for the selected subset of measurement channels, in which the co-registered geometry data represents the anatomy of the patient and spatial locations of the body surface electrodes in a common three-dimensional space. The method can repeat the selecting, storing and reconstructing for a plurality of subsets of electrodes to provide respective sets of reconstructed electrophysiological signals.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram that illustrates a system to generate electrocardiographic maps of cardiac electrical activity.

FIGS. 2, 3, 4 and 5 depict an arrangement of patches carrying respective electrodes distributed across a model of a patient's torso.

FIG. 6 is a block diagram that illustrates an example of an electrode geometry calculator.

FIG. 7 is a block diagram that illustrates another example of an electrode geometry calculator.

FIG. 8 is a block diagram that illustrates an example of a mapping system that includes reconstructed signal analysis.

FIG. 9 is a block diagram that illustrates an example of a mapping system that includes non-invasive signal analysis.

FIGS. 10A, 10B, 10C, 10D and 10E depict examples electrophysiological signals showing a comparison of signal waveforms, a set of patch electrodes and graphical maps provided for a full set of body surface electrodes and the set of patch electrodes.

FIGS. 11A and 11B depict examples of graphical potential maps for a cardiac envelope derived from signals measured by a full vest and patch electrodes, and FIG. 11C depicts EGM waveforms for a given point on a cardiac envelope on the cardiac envelopes of FIGS. 11A and 11B.

FIGS. 12A-17B are examples of different patch electrode configurations.

### DETAILED DESCRIPTION

This disclosure relates to using one or more patch-type electrodes for measuring electrophysiological signals from an outer surface of a patient's body. As described herein, the use of patch-type electrodes facilitates the workflow for performing ECGI and reduces the cost—both human and material costs—so ECGI can be used on a more widespread basis. As an example, the use of patch-type electrodes enables additional analyses to be performed on electrophysiological signals such as to identify input channels having a given sensitivity to low amplitude or other signal characteristics. As a result, information from such channels having low signal amplitudes or poor signal quality can be retained and analyzed, as described herein. This is in contrast to existing approaches where channels having low amplitudes or poor signal quality are usually removed from ECGI processing or otherwise diluted due to relative poor signal quality or low amplitude compared to other channels.

Additionally or alternatively, in some examples, systems and method disclosed herein can be configured to analyze reconstructed electrophysiological signals to identify one or more anatomical regions exhibiting a desired physiological characteristic. For example, reconstructed signals at respective anatomical locations on a cardiac envelope (e.g., the patient's heart) can be analyzed over time, such as in response to a surgical or other intervention. Signals exhibiting variations in response to the and/or during the intervention can be identified and emphasized (e.g., by weighting of respective signals) for re-computing the reconstructed signals on a cardiac envelope.

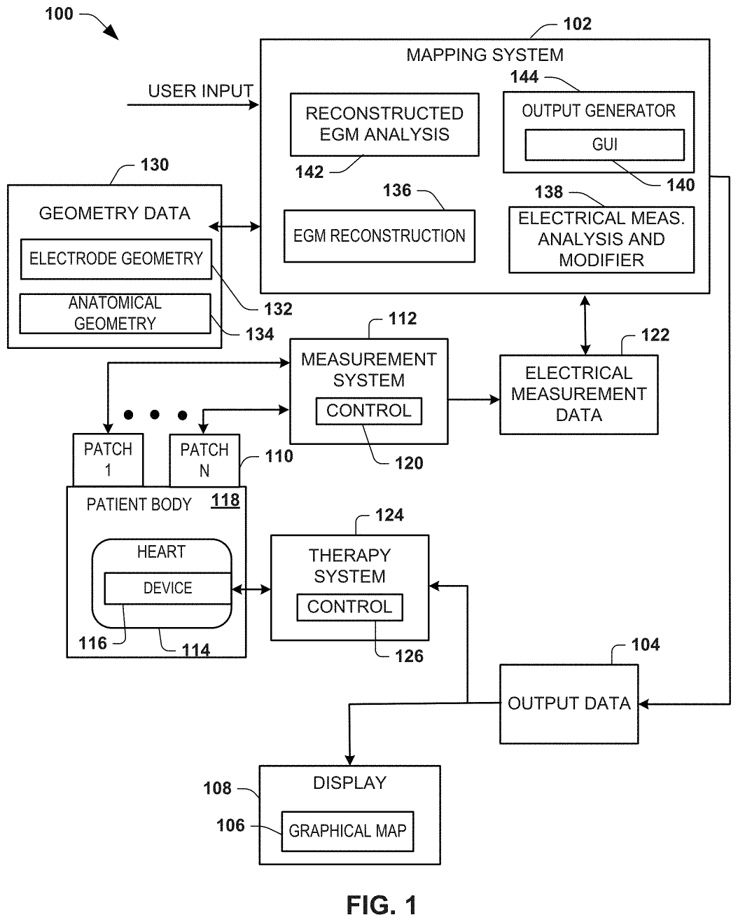

FIG. 1 is a block diagram that illustrates a system 100 for measuring and mapping electrophysiological activity, such as electrocardiographic maps of cardiac electrical activity for a patient's heart. For example, the system 100 includes a mapping system 102 configured to generate output data 104, which may be used to render a graphical map (e.g., a map on a heart model) 106 and/or display processed electrical signals on a display 108. The system 100 can also provide information in other formats to provide guidance to the user representative of and/or derived from electrical activity that may be measured non-invasively, invasively or a combination of invasively and non-invasively measured electrical signals.

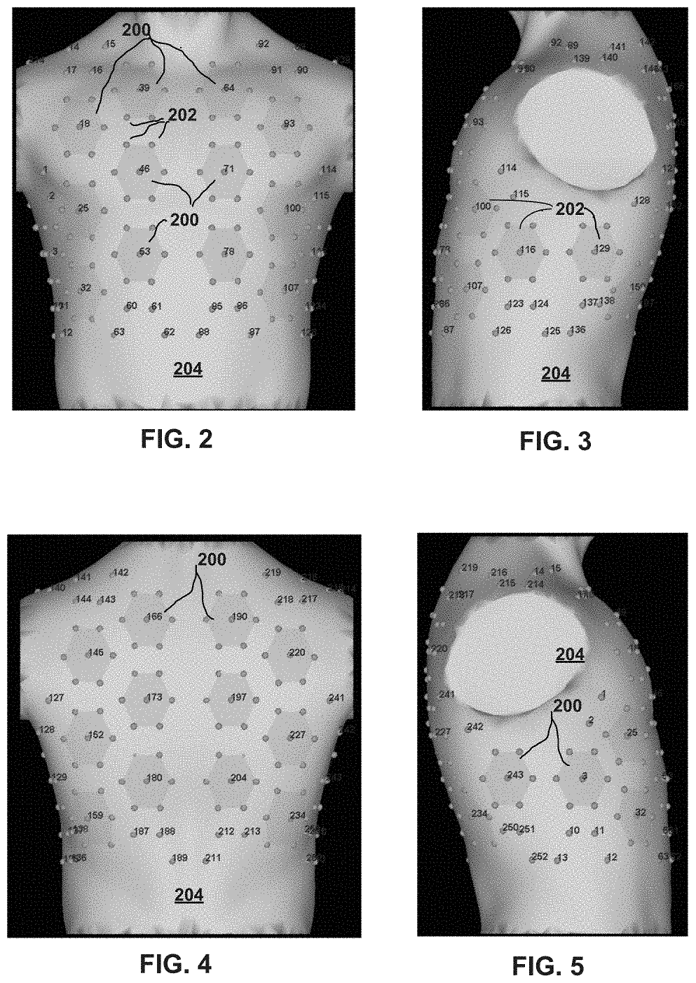

In the example of FIG. 1, the system 100 includes a plurality of patches 110, each having an arrangement of two or more electrodes configured to measure cardiac electrical activity non-invasively from an outer surface of the patient's body on which the patches are placed. For example, each patch 110 can include an arrangement of electrodes distributed across a web of flexible material (e.g., a panel or sheet) that is conformable to the contour of the patient's body surface where the patch is positioned. The size of the panel for each patch can vary depending on the number and spatial distribution of electrodes carried by the panel. For example, the patch can include a circular or rectangular arrangement of electrodes disposed on a web of flexible material, and the distribution can be uniform or non-uniform according to sensing requirements. An electrically conductive wire or other form of conductor can carry electrical measurement signals from electrodes on respective patches to a measurement system 112, or alternately, the signals can be transferred over optical fibers or wirelessly. Examples of patch-type sensors that could be used as the patches 110 in the system of FIG. 1 are shown in FIGS. 2-5, 10A-10B, and 12A-17B. Other patch-type sensors could be used in other examples based on this disclosure.

The measurement system 112 can include corresponding controls 120 configured to provide electrophysiological measurement data (also referred to herein as electrical data) 122 that describes electrophysiological activity (e.g., ECG signals) detected by electrodes of the respective patches 110. For example, signal processing circuitry (e.g., analog-to-digital conversion circuitry) of the measurement system 112 is configured convert measured analog measured electrophysiological signal to corresponding digital electrophysiological signals represented in the electrophysiological measurement data 122. The control 120 can also be configured to control the data acquisition process for measuring electrical activity and providing the measurement data 122 (e.g., at a predefined sampling rate).

As a further example, the patches 110 can be distributed over a portion of the patient's torso (e.g., thorax) to position respective electrodes at body surface locations for measuring electrical activity originating within the patient's heart 114. Because the patches are separate, a user can select and configure desired arrangements and numbers of patches 110 to position respective electrodes accordingly. For example, electrodes of the selected set of patches can cover the entire thorax. In another example, a set of patches may reside over a selected portion of the patient's torso (leaving one or more parts of the torso free of patches), such as designed for measuring electrical activity for a particular purpose (e.g., an array of electrodes specially designed for analyzing atrial fibrillation and/or ventricular fibrillation), to provide room for additional equipment and devices (e.g., defibrillator pads, 12-lead ECG or the like) and/or for monitoring a predetermined spatial region of the heart.

In some examples, the system 100 includes one or more sensors that may also be located on a device 116 that is adapted to be inserted into the patient's body 118 and to measure electrophysiological signals invasively, which can be provided to the measurement system 112 and stored as part of the electrical measurement data 122. For example, a catheter, having one or more therapy delivery devices 116 affixed thereto can be inserted into the body 118 as to contact the patient's heart 114, endocardially or epicardially. Various types and configurations of therapy delivery devices 116 can be utilized, which can vary depending on the type of treatment and the procedure. For instance, the therapy device 116 can be configured to deliver electrical therapy, chemical therapy, sound wave therapy, thermal therapy or any combination thereof.

By way of further example, the therapy delivery device 116 can include one or more electrodes located at a tip of an ablation catheter configured to generate heat or other energy form for ablating tissue in response to electrical signals (e.g., radiofrequency energy) supplied by a therapy system 124. In other examples, the therapy delivery device 116 can be configured to deliver cooling to perform ablation (e.g., cryogenic ablation), to deliver chemicals (e.g., drugs), ultrasound ablation, high-frequency radiofrequency ablation, pulsed field ablation, non-invasive ablation or a combination thereof. In still other examples, the therapy delivery device 116 can include one or more electrodes located at a tip of a pacing catheter to deliver electrical stimulation, such as for pacing the heart, in response to electrical signals (e.g., pacing current pulses) supplied by the therapy system 124. Other types of therapy can also be delivered via the therapy system 124 and the invasive therapy delivery device 116 that is positioned within the body.

As a further example, the therapy system 124 can be located external to the patient's body 118 and be configured to control therapy that is being delivered by the device 116. For instance, the therapy system 124 includes a control system (e.g., hardware and/or software) 126 that can communicate (e.g., supply) electrical signals via a conductive link electrically connected between the delivery device (e.g., one or more electrodes) 116 and the therapy system 124. The control system 126 can control parameters of the signals supplied to the device 116 (e.g., current, voltage, repetition rate, trigger delay, sensing trigger amplitude) for delivering therapy (e.g., ablation, stimulation, etc.) via electrode(s) of the therapy device 116 to one or more locations of the heart 114. The control system 126 can set the therapy parameters and apply stimulation based on automatic, manual (e.g., user input) or a combination of automatic and manual (e.g., semiautomatic controls). One or more sensors (not shown) of the device 116 can also communicate sensor information back to the therapy system 124. The position of the device 116 relative to the heart 114 can be determined and tracked intraoperatively via an imaging modality (e.g., fluoroscopy, X ray), a mapping system 102, direct vision, a localization system or the like. The location of the device 116 and the therapy parameters thus can be combined to determine corresponding therapy delivery parameters.

In each of such example approaches for acquiring electrical information from the patient's body 118, including invasively, non-invasively, or a combination of invasive and non-invasive sensing, the electrodes on the patches 110 provide the sensed electrophysiological information to the measurement system 112. In some examples, the control 120 can control acquisition of measurement data 122 separately from operation of the therapy system 124 (if implemented), such as in response to a user input. In other examples, the measurement data 122 can be acquired in coordination with (e.g., concurrently or otherwise synchronized with) delivering therapy by the therapy system 124, such as to detect electrical activity of the heart 114 that occurs in response to applying a given therapy (e.g., according to therapy parameters). For instance, appropriate time stamps can be utilized for indexing the temporal relationship between the respective measurement data 122 and other data that is used by the mapping system (e.g., therapy system 124) as to facilitate the evaluation and analysis thereof.

The mapping system 102 is programmed to combine the electrophysiological measurement data 122, corresponding to measured electrophysiological signals of the heart 114, with geometry data 130 to generate the output data 104. As described herein, for example, the output data 104 can represent or characterize electrophysiological signals on a surface of interest (e.g., a cardiac surface or other surface envelope on or within the heart 114). The mapping system 102 may further generate the output data 104 to represent information, including a representation of cardiac signals on a surface of interest, based on the electrophysiological measurement data 122 and the geometry data 130, as disclosed herein.

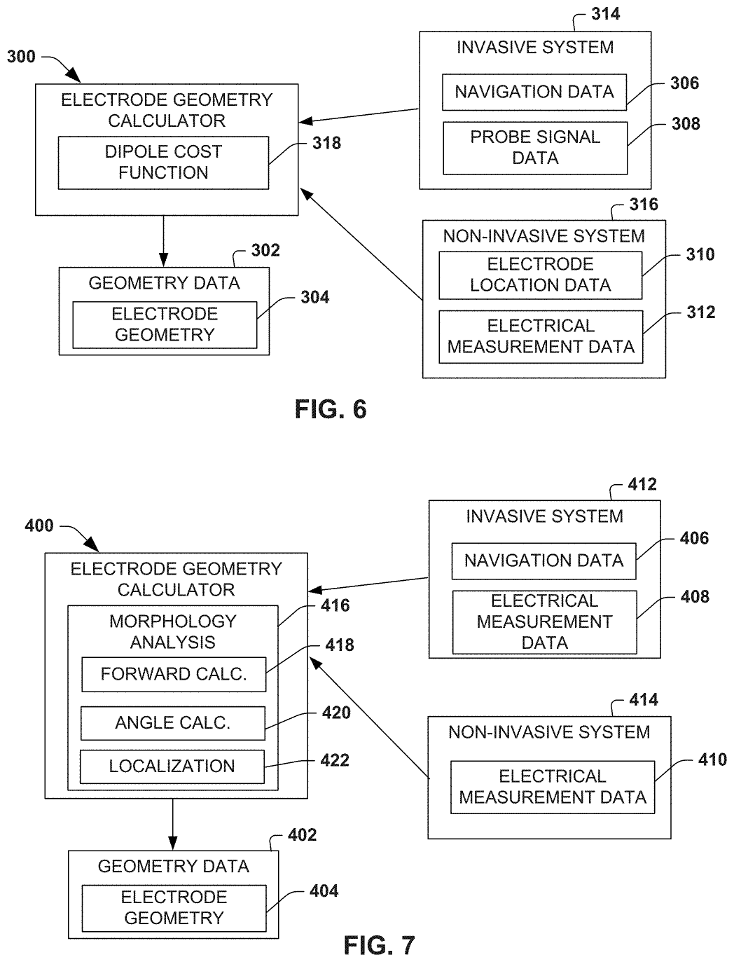

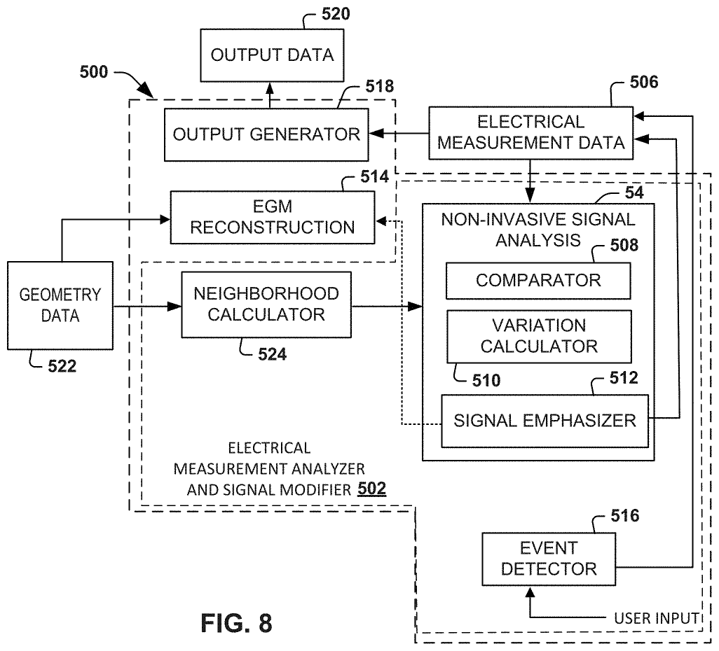

To enable the mapping system 102 to generate such output data, the geometry data 130 includes electrode geometry data 132 and anatomical geometry data 134. For example, the system 100 is further programmed derive the electrode geometry data 132 to represent locations of respective body surface electrodes in three-dimensional space based on (i) electrical signals measured by at least some of the electrodes on the patches 110, and/or (ii) a template describing the locations of respective body surface electrodes as applied to the patient's body 118. In another example, where electrodes are distributed across each of the patches 110 in a known spatial configuration and a spatial geometry of each of the patches is known (e.g., by localization) with respect to the patient's body 118 in a three-dimensional coordinate system, the electrode geometry data 132 can be readily extrapolated from the spatial geometry of the patches. In other examples, different approaches can be implemented to derive the electrode geometry data 132, such as described herein (see, e.g., FIGS. 6 and 7).

The anatomical geometry data 134 is generated to represent spatial geometry of the surface of interest of the patient in three-dimensional space. The anatomical geometry data 134 can be derived from imaging data acquired by a medical imaging modality and/or a user-specific template describing the anatomy of the patient. For example, an anatomical model can be constructed based on imaging data obtained (e.g., by a medical imaging modality) for the patient and provide spatial coordinates for the patient's heart 114 and, in some cases, the outer surface of the patient's body where the patches 110 are or will be positioned. The medical imaging data can be generated for the patient's body using a medical imaging modality, such as multi-plane x-ray, computed tomography (CT), magnetic resonance imaging (MRI), ultrasound, positron emission tomography (PET), single-photon emission computed tomography (SPECT) and the like. The electrode geometry data 132 and the anatomical geometry data 134 can be registered in a common three-dimensional spatial coordinate system.

In another example, the anatomical geometry data 134 may be provided by a template, such as including a look-up table (e.g., implemented as part of the mapping system 102 or as separate program code) in response to a user input. For example, the look-up table is configured as a template to provide an estimate of anatomical geometry data geometry in response to input data entered by the user describing one or more patient parameters. The patient parameters, for example, can include patient physical characteristics, medical information and the like, such as a combination of two or more of a patient body mass index, a measure of chest size, gender, height and weight, age (or other demographics), or known arrhythmias (e.g., determined from an ECG or other diagnostic tool). Additionally, in other examples, the anatomical geometry data 134 can be determined from imaging data acquired by a medical imaging modality individually or in combination with a user-specific template describing the physical characteristics of the patient.

As described above, the mapping system 102 is configured to generate output data 104, which can be used to render a graphical map 106 and/or present other information on the display 108. For example, the mapping system 102 includes an electrogram reconstruction function (e.g., machine-readable instructions) 136 programmed to compute an inverse solution and provide corresponding reconstructed electrograms across a surface of interest based on the electrophysiological measurement data 122 and the geometry data 130. The reconstructed electrograms thus can correspond to electrocardiographic activity across a surface of interest, such as a cardiac envelope, and can include static (three-dimensional at a given instant in time) and/or be dynamic (e.g., four-dimensional map that varies over time). Examples of inverse algorithms that can be implemented by the EGM reconstruction 136 include those disclosed in U.S. Pat. Nos. 7,983,743 and 6,772,004. The EGM reconstruction 136 can implement other inverse algorithms in other examples. The EGM reconstruction 136 thus is programmed reconstruct the body surface electrical activity measured via the sensor array 114 onto a multitude of locations on the surface of interest (e.g., greater than 1,000 locations, such as about 2,000 locations or more on a cardiac surface). Additionally or alternatively, the mapping system 102 can compute electrical activity over a sub-region of the heart based on electrical activity measured invasively, such as via a basket catheter or other form of measurement probe (e.g., on or attached to device 116).

In some examples, the mapping system 102 includes an electrical measurement analysis and modifier function (e.g., machine-readable instructions) 138. For example, the function 138 is programmed to modify values of electrophysiological signals for a subset of the electrodes (corresponding to respective input channels) to emphasize or de-emphasize respective signals measured over a time interval. In an example, the subset of electrodes (e.g., input channels) can be chosen to include electrodes that are part of one or more patches 110, and may be selected automatically or in response to a user input (e.g., user input provided by a graphical user interface (GUI) 140). In another example, the subset of electrodes (e.g., input channels) can be selected based on the function 138 analyzing one or more characteristics (e.g., amplitude) the signals. The function 138 can be programmed to perform a comparative analysis among all input channels to identify which channels exhibit a prescribed signal characteristic over one or more time intervals. Alternatively or additionally, the function 138 can perform a comparative multiple intervals for each respective signal channel to identify which channels exhibit changes in respective signals between two more time intervals, such as including low amplitude signal changes. The function 138 further can a machine learning model trained to determine the most likely subset of electrodes to optimize the solution for a given sensing application. For example, the machine learning model implemented by function 138 can be programmed to identify a configuration and arrangement of patch electrodes adapted to sense a user-specified type of arrhythmia (e.g., tachycardia, bradycardia, premature contractions, etc.). Additionally, or alternatively, the machine learning model implemented by function 138 can be programmed to identify a configuration and arrangement of patch electrodes adapted to sense electrophysiological signals at one or more regions of interest on the cardiac surface. For example, the machine learning model implemented by the function 138 can utilize one or more types of models, including support vector machines, regression models, self-organized maps, k-nearest neighbor classification or regression, fuzzy logic systems, data fusion processes, boosting and bagging methods, rule-based systems, artificial neural networks or convolutional neural networks.

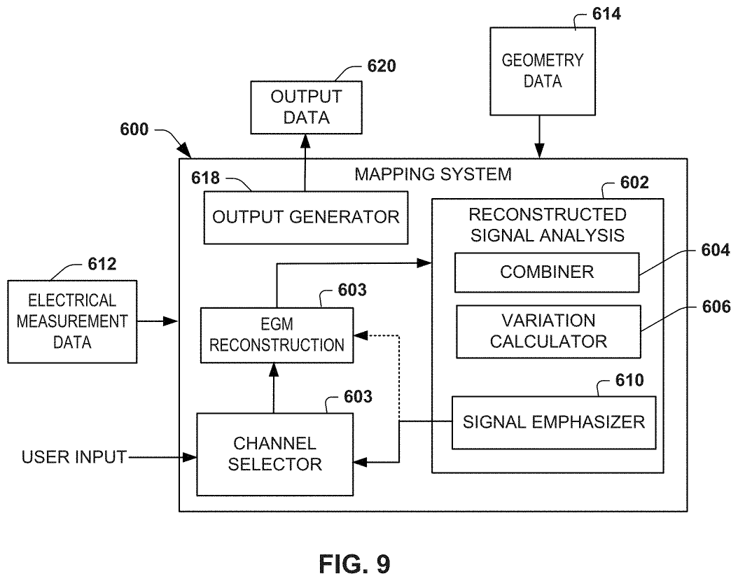

As a further example, the function 138 is programmed to modify electrophysiological signals so that signals for a subset of the channels in the electrical data are emphasized relative to the other channels. That is, the electrical data 122 is modified to represent emphasized versions of measured electrophysiological signals for the subset of channels and other (non-emphasized or normal) measured electrophysiological signals for other channels on the body surface. Thus, the reconstruction engine 136 can be programmed to compute reconstructed signals on the cardiac surface of interest (by solving the inverse problem) based on the co-registered geometry data 130 and modified electrical data 122, including the emphasized electrophysiological signals for the subset of channels. As a result of using emphasized signals during inverse reconstruction, the influence of the signals provided by the subset of channels can likewise be accentuated in the reconstructed electrograms. This can include programming the function 138 to accentuate a subset of channels having low amplitude signals (e.g., lower than a threshold) and/or channels exhibiting small signal changes or other signal characteristics over time that usually would be obfuscated by the influence of other (e.g., more pronounced) signals that are measured by electrodes distributed across the body surface. Additionally, or alternatively, the function 138 can be programmed to accentuate a subset of channels for signals having amplitudes exceeding a threshold) but exhibit changes that are obfuscated by the influence of a greater number of other channels other having signals that exhibit no change. Further information about function 138 is disclosed with respect to FIG. 8.

The mapping system 102 can also include a reconstructed EGM analysis and control function (e.g., machine-readable instructions) 142. In an example, the function 142 is programmed to select a proper subset of channels for the plurality of electrodes, and the EGM reconstruction function 136 calculates an inverse solution to reconstruct the electrophysiological signals on the cardiac envelope based on the subset of selected channels over one or more time interval. The functions 142 and 136 can repeat the process of selecting input channels and calculating the inverse solution for a plurality of different subsets of channels to provide respective sets of reconstructed electrophysiological signals over the same one or more time intervals. The proper subset of channels can be randomly or pseudorandomly selected from the plurality of electrodes. Alternatively or additionally, some or all channels can be selected for a subset in response to a user input (e.g., using GUI 140) selecting which channels to include or exclude from a given channel subset. The number of channels selected can be the same for each subset or different numbers of channels can be used, such as ranging from one to a subset that includes all channels. Because a different subset of channels are used to generate each respective set of reconstructed electrophysiological signals (over the same time interval(s)), each computed set of reconstructed electrophysiological signals can represent electrical activity across the same surface of interest (the entire heart or a region of interest) in manner that can vary based on different arrangements of channels influencing the resulting reconstructed electrophysiological signals when the inverse solution is calculated. As a result, the electrogram reconstruction function 136 can generate different sets of reconstructed electrophysiological signals for a given time interval based on non-invasive electrical measurement data for different subsets of channels over the given time interval. The results reconstructed electrophysiological signals thus can show respective conditions or characteristics across the patient's heart and/or for a particular region of interest that can vary according to the relative influence of the respective input channels used for each reconstruction. For example, a number of patches having associated electrodes and the arrangement of such patches thus can be placed on the outer surface of a patient's body particularly adapted to sense a specific arrhythmia condition (e.g., atrial fibrillation, premature ventricular contraction (PVC), atrial tachycardia, ventricular tachycardia, or the like) for a given patient.

Because the patches and electrodes can be customized in this or another manner consistent with this disclosure, fewer patches and electrodes can be used in certain circumstances without reducing (and possibly increasing) the signal-to-noise ratio (SNR). The reduction in the number of electrodes further can reduce the overall cost to the patient.

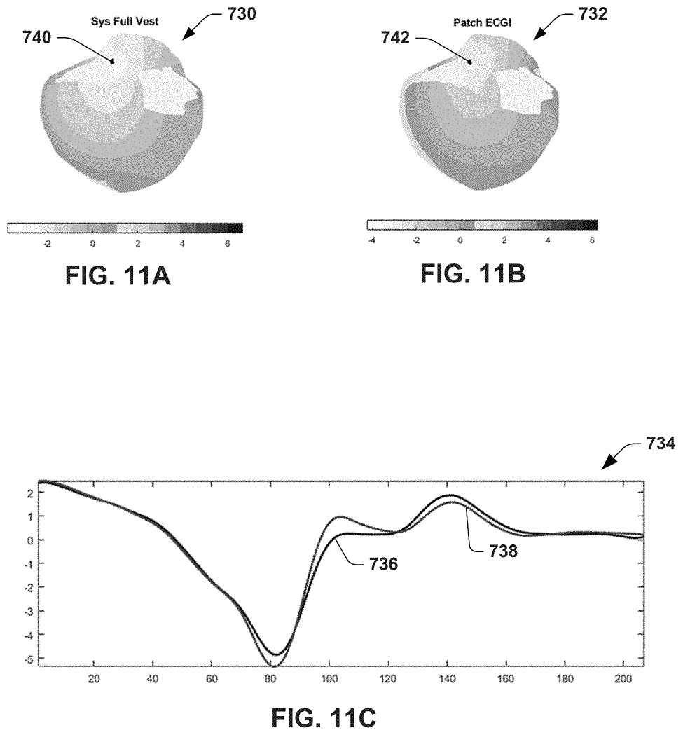

The mapping system 102 also includes an output generator 144 to provide the output data 104 to visualize a graphical map 106 or other information on the display 108 based on measurement data 122 and the geometry data 130 for one or more time intervals. Some examples of output displays that can be provided by the output generator 144 include graphical representations of measured electrophysiological signals based on the data 122 and/or graphical maps of reconstructed electrophysiological signals, such as disclosed with respect to FIGS. 10D-10E. The output generator 144 may also generate the output data 104 to display other types of information, such as time-domain plots, frequency-domain plots or the like. As disclosed herein, the graphical maps and other information can be computed based on electrical data that is acquired non-invasively via one or more electrodes located on patches 110 distributed across the surface of the patient's body 118. The time interval for which the output data/maps are computed can be selected based on user input, such as via the GUI 140. Additionally or alternatively, the selected time intervals can be detected and synchronized with the application of therapy by the therapy system 124.

Additionally, the output data 104 can be utilized by the control 126 of the therapy system 124 in examples that include the therapy system in the system 100. For example, the therapy control 126 can be fully automated control, semi-automated control (partially automated and responsive to a user input via GUI 140) based on the output data 104 or manual control. In some examples, the control system 126 for the therapy system 124 can utilize the output data 104 to control one or more therapy control parameters. As an example, the control 126 can control delivery of ablation therapy to a site of the heart (e.g., epicardial or endocardial wall) based on the analysis of electrical measurement data (by function 138) or the analysis of reconstructed electrophysiological signals (by function 142), such as disclosed herein. For instance, the delivery of therapy can be terminated automatically in response to detecting the absence of a previously detected arrhythmogenic condition. In other examples, an individual user can view the map generated in the display to manually control the therapy system based on information in the graphical map 106 that is visualized on the display 108. Other types of therapy and devices can also be controlled based on the output data 104.

As a further example, the system 100 has applications throughout various phases of patient care. As an example, the system can be used as part of a patient screening process, such as part of a patient risk stratification process (e.g., part of a diagnostic and/or treatment planning procedure). Additionally, the system 100 can be utilized as part of a treatment procedure, such as to determine parameters for delivering a therapy to the patient (e.g., delivery location, amount and type of therapy by one or more therapy system 124). The system 100 further may be used to perform post-treatment evaluation of the patient.

FIGS. 2, 3, 4, and 5 show different views of patches 200 having respective electrodes 202 distributed across an outer surface of a patient's thorax 204. In particular, FIG. 2 is a front view, FIGS. 3 and 5 are side views, and FIG. 4 is a back view. In the example of FIGS. 2, 3, 4, and 5, each of the patches is shown as having a generally circular shape and are spaced evenly across the thorax 204 so as to position the electrodes 202 uniformly across the thorax. As shown, each patch 200 can include seven electrodes 202, including one central electrode and six along a periphery of the patch spaced a common distance from the central electrode. Different numbers and arrangement of electrodes 202 can be used in other examples. Also shown in FIGS. 2, 3, 4, and 5 a number of free electrodes 202 are positioned on the thorax 204, which free electrodes are not attached to or implemented on respective patches. Free electrodes can be used to place electrodes at one or more respective locations where it is desired to position fewer electrodes than a given patch includes. In other examples, different configurations of patches can be used, in which each patch configuration has a respective different number of electrodes, but may include a common distribution and arrangement of electrodes. In another example, different patch configurations can have include a different distribution and arrangement of electrodes, such as to provide application-specific patch configurations. In each configuration, the relative spatial position and geometry of electrodes across each respective patch is known. Thus, by knowing or determining a relative spatial position of a given patch on a patient's body, the spatial position of respective electrodes on the patient's body can likewise be known or readily determined and stored as electrode geometry data (e.g., data 132).

FIG. 6 is a block diagram showing an example of an electrode geometry calculator 300 programmed to provide geometry data 302, including electrode geometry data 304. The electrode geometry data 304 can correspond to electrode geometry data 132 implemented in the system 100 of FIG. 1, and the description of FIG. 3 also refers to FIG. 1.

In an example, the electrode geometry calculator 300 is programmed to determine the electrode geometry data based on navigation data 306, probe signal data 308, electrode location data 310 and non-invasive electrical measurement data 312. For example, an invasive system 314 is configured to provide the navigation data 306 and probe signal data 308. The navigation data 306 can represent spatial coordinates (e.g., three-dimensional coordinates) of a probe that is positioned within the patient's body. A non-invasive system 316 is configured to provide the electrode location data 310 and electrical measurement data 312. In some examples, the probe signal data (e.g., representing parameters of an applied waveform) 308 and the electrode location data (e.g., representing a spatial relationship among electrodes) 312 can be fixed and known a priori.

As an example, a navigation system (e.g., part of invasive system 314) can be configured to provide the navigation data 306 to represent the position of a probe within a patient's body. Examples of cardiac navigation systems that can be implemented to provide the navigation data include the EnSite NavX navigation and visualization system (commercially available from Abbott), the CARTO cardiac mapping system (commercially available from Johnson & Johnson), the NOGA XP cardiac navigation system (commercially available from Biosense Webster) to name a few. Other navigation system may be used within the invasive system 314 in other examples to provide the navigation data 306. In yet another example, the navigation data 306 can represent a known location within the patient's body, such as an anatomical landmark, at which a probe is positioned when providing a probe signal.

As a further example, the probe signal data 308 can represent an applied signal that is provided by one or more electrodes disposed on a probe, such as a catheter or other device that is adapted to be inserted into the patient's body. For example, the probe signal data can include parameter describing the applied signal (e.g., signal morphology, such as waveform shape, duty cycle frequency) as well as a time parameter (e.g., time stamp) when the signal is applied. The electrode is configured to provide an applied electrical signal within the patient's body. If more than one electrode used to provide the prove signal data 308, each such electrode is at predetermined location relative to each other. A signal generator can apply a specific signal to the probe electrode, which can be measured by electrodes on the body surface (e.g., patch electrodes), as described below. For example, the applied signal can be a predetermined waveform, such as may be a pulse, a square wave, a sinusoidal waveform or the like that can be generated by a signal source electrically connected to the electrode and is distinguishable from anatomically generated signals. The navigation data 306 represents the spatial location of the probe, including at the time when the probe provides the probe signal. For example, the navigation data 306 and probe signal data 308 can include a time stamp to enable the electrode geometry calculator 300 to determine the location of the probe when the probe provides the probe signal.

As mentioned, the non-invasive system 316 is configured to provide the electrode location data 310 and electrical measurement data 312. For example, the non-invasive system includes electrodes, such as can be on one or more patches, arranged on an outer surface of the patient's body to measure body surface electrophysiological signals corresponding to the electrical measurement data 312. As described herein, the relative spatial position of electrodes on each patch 110 is known and can be stored in memory as the electrode location data 310.

As a further example, the electrode geometry calculator 300 includes a dipole model cost function 318 having parameters representing a dipole location and moment corresponding to the applied electrical signal, as described by the probe signal data. The geometry calculator 300 is programmed to apply a boundary condition on the dipole model cost function 318 to determine a location of one or more of the electrodes relative to a known location of the probe and to generate the electrode geometry data 304 for such one or more electrodes based on the determined location of the electrodes. Thus, by determining a spatial location of one electrode on a given patch 110 according to the dipole model cost function, the geometry calculator 300 can determine the spatial location of each other electrode on the given patch based on the electrode location data 310 for the given patch. Alternatively, the geometry calculator 300 can be programmed to employ the dipole model cost function 318 to determine the spatial location of each electrode independently of the electrode location data 310. An example of a dipole model cost function, which can be implemented as the dipole model cost function 318, is disclosed in U.S. Patent Publication No. 2016/0061599.

Advantageously, the electrode geometry calculator 300 can determine the electrode geometry 304 without requiring imaging of the patient while the electrodes are positioned on the patient's body. This allows the application of electrodes to be independent of access to an imaging modality, which can significantly increase the use of the systems and methods disclosed herein. By not requiring use of a medical imaging modality, the overall cost of the process can likewise be reduced.

FIG. 7 is a block diagram showing another example of an electrode geometry calculator 400 that can be implemented to provide geometry data 402, including electrode geometry data 404. For example, the electrode geometry calculator 400 can be programmed to provide the electrode geometry data 404, which corresponds to electrode geometry data 132 implemented in the system 100 of FIG. 1. Accordingly, the description of FIG. 4 also refers to FIG. 1.

Similar to FIG. 7, the electrode geometry calculator 400 is programmed to determine the electrode geometry data 404 based on navigation data 406, invasive measurement data 408, and non-invasive electrical measurement data 410. For example, an invasive system 412 is configured to provide the navigation data 406 and invasive measurement data 408. The navigation data 306 can represent spatial coordinates (e.g., three-dimensional coordinates) of a probe that is positioned within the patient's body, including the real-time position of the probe, as disclosed herein. The navigation data 406 can also include a time stamp to identify a time that is associated with each measurement of probe position (e.g., made by a navigation system).

The probe includes one or more electrodes configured to measure electrophysiological signals on or within the patient's heart and provide the electrical measurement data 408 to represent a measured intrabody electrophysiological signal at a known location within the patient's body. Each measurement location is known based on the navigation data 406. The probe can be a contact probe or a non-contact probe configured to measure the electrophysiological signals accordingly. The measured electrophysiological signal may be a unipolar or bipolar signal, which can vary depending on the configuration and arrangement of electrodes on the probe. The electrical measurement data 408 can include signal data representing the amplitude of a measured electrical signal over time and time stamp data to identify the time of respective measurements. The time stamps of the navigation data and the electrical measurement data 408 can by synchronized or otherwise coded to enable the respective data to be synchronized.

A non-invasive system 414 is configured to provide the electrical measurement data 410. As described herein, the non-invasive system 414 includes an arrangement of electrodes adapted to be positioned on the outer surface of a patient's body, such as implemented on one or more patches 110. For example, the non-invasive system can include measurement system 112 configured to measure body surface electrophysiological signals based on signals sensed by the electrodes and provide the corresponding electrical measurement data 410, which includes a time stamp or other indication of time associated with the measured electrophysiological signals.

The electrode geometry calculator 400 includes morphology analysis function 416 programmed to analyze morphology of signals based on the intrabody electrical measurement data 408 and the non-invasive electrical measurement data 410, including the measured non-invasively by the body surface electrodes. The electrode geometry calculator 400 further is programmed to determine the electrode geometry data 404 based on the morphological signal analysis. For example, the morphology analysis function 416 is programmed to compare a morphology of the measured intrabody electrophysiological signal(s) with a morphology of the respective signals measured on the patient's body to generate the electrode geometry data based on the comparison.

As a further example, the morphology analysis function 416 includes a forward calculation function 418 programmed to compute a forward calculation to determine a representation of the measured intrabody electrophysiological signal on the outer surface of the patient's body. The forward calculation function 418 can be programmed to determine electrical signals on the outer surface of the patient's body based on the measured electrical signal at the known intrabody location. For example, the forward calculation function 418 employs the Laplace equation to relate measurements at each body surface electrode location and the known intrabody location, in which the location of the body surface electrodes are unknown parameters. The known intrabody location can be on the septum of the heart or another known location. The forward calculator thus can compute the electrophysiological signals on the body surface at respective angles on the body surface with respect to the known intrabody location.

For example, assuming spatial geometries from torso and heart already segmented from CT or MRI, but patch electrode/vest may not be placed on the patient during imaging during CT/MRI. Given an R wave from normal sinus rhythm, the activation pattern is a strong dipole moving fast along ventricle septum. The duration of the R wave can be known, and the dipole direction and location also can be known based on heart anatomy. The forward calculation function 418 can thus be programmed to forward calculate the dipole to torso surface and create pseudo ECG tracings projected onto the body surface for one or more regions of interest. ECG signals can be measured for each of patches placed on the patient's body surface, and the measured ECG signals can be matched with the projected pseudo ECGs (e.g., using correlation or other matching algorithm). The region having the best match can be identified as the actual location of the patch, and the location information can be stored in memory and/or displayed graphically. The overall process implemented by morphology analysis function 416 can be an iterative process, so that the region of forward calculation (e.g., computed by forward calculation function 418) can be refined over a set of iterations to determine the location with improved precision (e.g., higher resolution).

The morphology analysis function 416 also includes an angle calculator 420 programmed to calculate angle from the known intrabody location within the patient's body to respective electrodes. For example, the angle calculator 420 can be programmed to implement a minimization algorithm (e.g., least squares minimization or the like) to determine the respective angles for each electrode location. In another example, the angle calculator 420 can be programmed to determine respective angles by employing a brute force method or other optimization method. The morphology analysis function includes a localization function 422 programmed to determine the location of the respective electrodes to provide the electrode geometry data 404 based on the respective calculated angles for the electrodes computed from the forward calculation.

FIG. 8 is a block diagram showing an example of a mapping system 500 that includes an electrical measurement analyzer and signal modifier function 502. The function 502 provides an example of a method (e.g., executable instructions) that can be used to implement the function 138 of FIG. 1. Accordingly, the description of FIG. 8 also refers to FIG. 1.

As shown in the example of FIG. 8, the function 502 includes a non-invasive signal analysis function 504 programmed to analyze electrophysiological signals provided in electrical measurement data 506. For example, the electrical measurement data corresponds to electrical data 122 representing measured electrophysiological signals provided from a plurality of electrodes distributed across an outer surface of a patient's body. Each electrode, electrical connectors (wires and/or traces), and/or input signal processing circuitry (e.g., amplifiers and filters of measurement system 112) can define a respective input channel that provides a measured electrophysiological signal for a respective location on the body surface. In an example, the electrodes are implemented on one or more patches adapted to be placed on the outer surface of the patient's body and sense cardiac electrical activity, such as described herein.

The analysis function 504 can include a comparator function 508 programmed to compare electrophysiological signals. The comparator function 508 can compare signals of different signal channels over the same time interval (or time instance) or compare signals for the same signal channels across two more different time intervals (or time instances). In another example, the comparator function 508 can compare each of the signal channels (or a selected subset of channels) to a baseline, which can be a normalized baseline or a patient-specific baseline. The comparator function 508 can compare different signal parameters, such as a comparison of signal amplitude, frequency and/or morphology. The type of signal analysis and/or the signals being analyzed by the analysis function 504 can be preprogrammed (e.g., by default) or set by a user (e.g., in response to a user input). Additionally or alternatively, the comparator function 508 can be programmed to perform a comparative analysis among all input channels to identify which channels exhibit a prescribed signal characteristic over one or more time intervals. Alternatively or additionally, the comparator function can perform a comparative analysis for each respective signal channel over time to ascertain which channels exhibit changes in respective signals between two more time intervals, such as including low amplitude signal changes.

As a further example, the variation calculator 510 is programmed to compute an indication of variation of the electrophysiological signals. The variation can depend on the type of comparative analysis being implemented by the comparator function. For example, the variation calculator 510 can compute the variation in signal magnitude, such as representing a difference between signal values, such as signal amplitude (e.g., a difference between average amplitude, maximum amplitude or dominant frequencies) over a time interval. Alternatively, the variation calculator 510 can compute the variation as statistical variance or standard deviation from a mean signal value (e.g., signal amplitude and/or frequency). The determined variation for each channel can be stored in memory linked to the respective channel for additional signal processing functions, such as disclosed herein.

The function 502 also includes a signal emphasizer 512 that is programmed to modify values of electrophysiological signals for a subset of the electrodes (corresponding to respective input channels). For example, the signal emphasizer 512 can be programmed to emphasize or de-emphasize respective signals (or features of such signals) measured over a time interval. The signal emphasizer 512 can be programmed to modify signals for the subset of electrodes (e.g., input channels) based on a spatial relationship of the electrodes. For example, the signal emphasizer 512 can be programmed to select electrodes that are part of one or more patches 110, and may be selected automatically or in response to a user input (e.g., user input provided by a graphical user interface (GUI) 140). In another example, the signal emphasizer 512 can be programmed to select the subset of electrodes (e.g., input channels) based on the analysis implemented by the signal analysis function 504, including the comparator function 508 and/or the variation calculator 510. Additionally, regardless of how the subset of electrodes is selected, the signal emphasizer 512 can be programmed to modify the signal values of the selected subset of channels based on the analysis implemented by the signal analysis function 504.

As a further example, the signal emphasizer 512 is programmed to modify electrophysiological signals so that signals (or certain signal features) for the selected subset of the channels in the electrical data 506 are emphasized (e.g., by increase signal amplitude) relative to other channels. In an example, the signal emphasizer 512 emphasizes a given channel in the selected subset of channels by weighting the given channel so that the relative amplitude of the given signal is increased. The signal emphasizer 512 can apply a uniform (e.g., the same) weighting to signals in each of the selected subset of channels. In another example, signal emphasizer 512 can apply a variable weighting to respective signals in each of the selected subset of channels, which weighting can be set based on the signal analysis by function 504. Additionally, the weighting may further vary over time such as based on the signal analysis, including the analysis implemented by the comparator function 508 and the variation calculator 510. While the modification is described as increasing the emphasizing the selected subset of channels, in other examples, this can be implemented by de-emphasizing the non-selected remaining subset of channels so that the net relative value of the selected subset of channels are increased.

In an example, the signal emphasizer 512 is programmed to modify the electrical measurement data 506, which is stored in memory, to represent emphasized versions of measured electrophysiological signals for the subset of channels and leave alone (e.g., not emphasize) measured electrophysiological signals for the other channels for electrodes on the body surface. In another example, the signal emphasizer 512 is programmed to implement weighting of the channels to be applied by an electrogram reconstruction function 514 as part of solving the inverse problem. For example, the weighting can be implemented as a multiplying factor for elements of a transfer matrix that relates the geometry between the heart and body surface where the electrodes are position. As one example, measurement in each row can be scaled by multiplying the measurement by a respective weight value (e.g., w₁ though w_{N}), such as follows:

$$[ \begin{matrix}{w}_{1} & {w}_{1} f ( {x}_{1} - {y}_{1} ) & … & {w}_{1} f ( {x}_{1} - {y}_{M} ) \\ {w}_{2} & {w}_{2} f ( {x}_{2} - {y}_{1} ) & … & {w}_{2} f ( {x}_{2} - {y}_{M} ) \\ ⋮ & ⋮ & ⋮ & ⋮ \\ {w}_{n} & {w}_{n} f ( {x}_{N} - {y}_{1} ) & … & {w}_{n} f ( {x}_{N} - {y}_{M} ) \\ 0 & \frac{{w}_{1} \partial f ( {x}_{1} - {y}_{1} )}{\partial \vec{n}} & … & \frac{{w}_{1} \partial f ( {x}_{1} - {y}_{M} )}{\partial \vec{n}} \\ 0 & \frac{{w}_{2} \partial f ( {x}_{2} - {y}_{1} )}{\partial \vec{n}} & … & \frac{{w}_{2} \partial f ( {x}_{2} - {y}_{M} )}{\partial \vec{n}} \\ ⋮ & ⋮ & ⋮ & ⋮ \\ 0 & \frac{{w}_{n} \partial f ( {x}_{N} - {y}_{1} )}{\partial \vec{n}} & … & \frac{{w}_{n} \partial f ( {x}_{N} - {y}_{M} )}{\partial \vec{n}}\end{matrix} ] [ \begin{matrix}{a}_{0} \\ {a}_{1} \\ \\ . \\ \\ . \\ \\ . \\ \\ {a}_{m}\end{matrix} ] = [ \begin{matrix}{w}_{1} u ( {x}_{1} ) \\ {w}_{2} u ( {x}_{2} ) \\ ⋮ \\ {w}_{n} u ( {x}_{N} ) \\ 0 \\ 0 \\ . \\ . \\ . \\ \\ \\ 0\end{matrix} ]$$

Thus, the reconstruction function 514 can be programmed to compute reconstructed signals on the cardiac surface of interest (by solving the inverse problem) based on the co-registered geometry data 522 and modified electrical measurement data 506, including the emphasized electrophysiological signals for the subset of channels. As a result of using emphasized (and/or de-emphasized) signals during inverse reconstruction, the influence of the signals provided by the subset of channels can be accentuated in the reconstructed electrograms. This can include programming the signal emphasizer 512 to accentuate a subset of channels having low amplitude signals (e.g., lower than a threshold) and/or channels exhibiting small signal changes or other signal characteristics over time that usually would be obfuscated by the influence of other (e.g., more pronounced) signals that are measured by electrodes distributed across the body surface.

By way of further example, the comparator function 508 can be programmed to compare electrophysiological signals (provided in the electrical measurement data) 506 measured by the electrodes (e.g., implemented on patches) positioned on the body surface relative to baseline data for the same patient measured on the body surface over one or more different time intervals. The comparator function 506 can be programmed to identify (e.g., by tagging or flagging) the subset of the channels exhibiting small signal changes from the baseline signal values. In an example, the baseline data represents electrophysiological signals prior to an intervention event, and the detected changes represent electrophysiological signals measured at a time during or after the event. Examples of interventions can include chemical, electrical stimulation, thermal (e.g., cryogenic or heat) ablation, radiation, radiofrequency, laser, non-contact pulse field or other therapy applied to the heart. In an example, the variation calculator 510 can be programmed compare the detected change in the measured signals relative to a threshold to ascertain which subset of channels exhibits a greater relative change compared to the threshold. The same threshold can be applied to signal changes determined for each of the respective channels or different thresholds can be used, such as regionally or on a per channel basis.

In an example, the system 500 includes an event detector 516 programmed to detect the event and to store an indication of the event, such as a time or time interval during which the event occurred. The non-invasive signal analysis function 504 thus can employ the stored event indication to select baseline or pre-event electrical measurement data at a time prior to the event, and to select intra-event or post-event electrical measurement data 506 during or after the event, respectively. The signal analysis function 504 (e.g., including the comparator function 508 and variation calculator 510) can then perform corresponding signal analysis with respect to the selected pre-event and intra- and/or post-event electrical measurement data 506, as disclosed herein. As described herein, for example, the signal emphasizer can be programmed to emphasize the subset of channels by increasing the weight the subset of channels exhibiting the greater relative changes and removing and/or decreasing the weight of channels exhibiting smaller changes with respect to the detected event. Alternatively, where a user desires to emphasize signals exhibiting smaller changes relative to an event, the signal emphasizer can be programmed to de-emphasize the subset of channels exhibiting the greater relative changes and increasing the weight of channels exhibiting smaller changes with respect to the detected event. The reconstruction function 514 thus is programmed to generate reconstructed electrograms according to the weighting or other adjustments applied to the electrical measurement data 506 and based on the geometry data 522.

As described herein, an output generator 518 is programmed to provide the output data 520 to visualize a graphical map or other information on a display (e.g., display 108) based on the reconstructed electrograms generated for one or more time intervals. One or more graphical maps may be displayed concurrently on the display, such as including a map without weighting applied and with one or more maps in which weighting (e.g., different weighting) has been applied by signal emphasizer 512. The different maps being displayed can be for the same time interval. Alternatively, the different maps being displayed can be for different intervals, such as including a time interval before a given event and a time interval after the event.

In some examples, the system 500 further includes a neighborhood calculator 524 programmed to determine a proximity of electrodes on the patient's body based on a spatial analysis of the electrode geometry data, which is part of the geometry data 522. The electrode geometry data can be determined according to any approach disclosed herein (see, e.g., FIGS. 1, 6 and/or 7). The signal analysis function can be programmed to evaluate electrophysiological signals measured by electrodes in each respective spatial neighborhood, as specified by the neighborhood calculator. For example, the neighborhood calculator 524 can assign each of the spatial neighborhoods to include electrodes of a respective patch 110, a set of two or more adjacent patches 110 or an arrangement of neighboring electrodes located on two or more patches 110. The neighborhood calculator 524 can group electrodes together into respective spatial neighborhoods automatically or in response to a user input (e.g., using GUI 140) selecting which patches 110 and/or respective electrodes to include a given spatial neighborhood. In a further example, the signal analysis function 504 can be programmed to apply the comparator function 508 and/or the variation calculator 510 to analyze signal channels for electrodes within each spatial neighborhood.

In an example, the electrophysiological signal measurements are made by electrodes on the body surface with respect to a common reference. The reference can be a global reference for all electrophysiological signal measurements. Alternatively, each region (e.g., neighborhood) can have a respective reference that is set, such as at a centroid of the region or selected in response to a user input.

For example, the signal analysis function 504 is programmed (e.g., by applying the comparator function 508 and/or the variation calculator 510) to analyze electrophysiological signal measurements with each neighborhood (e.g., 2-10 cm² diameter region on a cardiac surface), as identified by the neighborhood calculator 524, for changes. Alternatively, the neighborhood calculator 524 can specify a given region as defined by a given patch, such that the signal analysis function 504 analyzes electrophysiological signals for the given patch for changes. The signal analysis function 504 can be programmed to determine the amount of signal change (e.g., low amplitude changes) for each region, and signal emphasizer 512 can apply weighting to respective channels if the signal change for the associated neighborhood exceeds a delta/threshold.

In some examples, the signal emphasizer 512 is programmed to further weight regions (e.g., respective spatial neighborhoods) with a weighting factor that is a function of (e.g., be proportional to) an amount of change (e.g., determined by the analysis function 504 relative to common reference) that each of the spatial neighborhoods exhibits. For example, the emphasizer 512 can weight a given neighborhood of signal channels exhibiting a greater amount of change in electrophysiological signals relative to signals channels of one or more other neighborhoods more heavily (e.g., being multiplied by a larger weighting factor) than the other neighborhoods exhibiting a lesser amount of relative change in the measured electrophysiological signals. In another example, neighborhoods exhibiting a lesser amount of relative change in the measured electrophysiological signals may be non-weighted or even may be de-emphasized (or removed altogether) by applying a fractional weighting that is less than one (e.g., <1.0). In this way, the relative influence of spatial regions exhibiting more variation can be increased over other regions, and the resulting output data 520 can be used to visualize corresponding signal activity in graphical maps of the reconstructed electrophysiological signals. For instance, this approach enables a user to observe subtle changes in electrophysiological measurements during an intervention as well as from comparison of before and after the intervention. That is, the weighting can be spatially correlated with exhibiting subtle changes so that the channels with subtle changes are emphasized and stand out more in the output data 520 and are displayed in associated graphical maps.

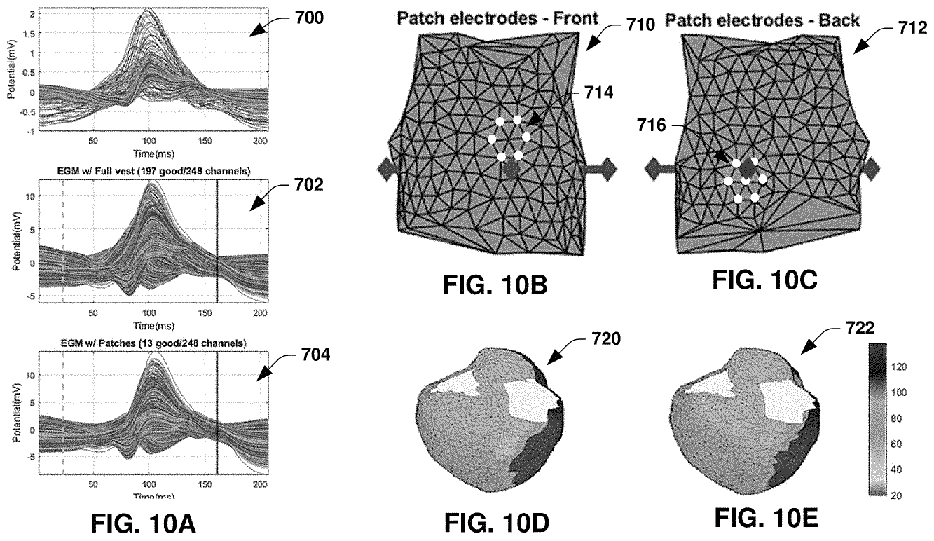

FIG. 9 is a block diagram showing an example of a mapping system 600 that includes a reconstructed signal analysis and modifier function 602. The function 602 provides an example of a method (e.g., executable instructions) that can be used to implement the function 142 of FIG. 1. In some examples, the reconstructed signal analysis and modifier function 602 also can be used in a mapping system in combination with the non-invasive signal analysis and modifier function 502 of FIG. 8. Accordingly, the description of FIG. 9 also refers to FIGS. 1 and 8.

In the example of FIG. 9, a channel selector 603 is programmed to select different subsets of channels for respective electrodes that are to be used from the electrical measurement data 612 for electrogram reconstruction. The channel selector 603 can randomly or pseudorandomly select from the plurality of channels. Alternatively, or additionally, the channel selector 603 can be programmed to select one or more subset of channels responsive to a user input. An electrogram reconstruction function 608 (e.g., corresponding to electrogram reconstruction function 136) generates respective sets of reconstructed electrophysiological signals for one or more time intervals for each selected subset of channels based on corresponding electrical measurement data 612 and geometry data 614. The time interval(s) for use by the electrogram reconstruction function 608 can be also be selected in response to a user input or through an automated interval selection method. The process of selecting and reconstructing electrophysiological signals for different subsets of electrodes for a given time interval thus can be repeated to provide a respective set of reconstructed electrophysiological signals for each selected subset of channels to seek convergence toward a unified solution.

The reconstructed signal analysis and modifier function 602 thus is programmed to analyze the subsets of reconstructed electrophysiological signals. The reconstructed signal analysis and modifier function 602 includes a combiner function 604 and a variation calculator 606. The reconstructed signal analysis and modifier function 602 is programmed to analyze one or more sets of reconstructed electrophysiological signals. The analysis can be across the entire surface of interest onto which the electrophysiological signals have been reconstructed or the analysis can be constrained to a selected region of interest (e.g., selected in response to a user input).

In an example, the combiner function 604 can be programmed to combine (e.g., average together) electrophysiological signals that have been reconstructed (by electrograms reconstruction function 608) across the surface of interest from two or more respective sets of reconstructed electrophysiological signals to provide to provide an aggregate set of reconstructed electrophysiological signals. For example, the combiner function 604 can average (or add together) signal values at each point (e.g., node) distributed across the surface of interest (e.g., an epicardial surface) over a time interval(s) to generate the aggregate set of reconstructed signals. More than one aggregate set of reconstructed electrophysiological signals can be generated for a given time period, which may be further analyzed and re-combined (e.g., by function 602) based on the results of such analysis.

The variation calculator 606 may be implemented as an instance of the variation calculator 510 of FIG. 8, such as applied and programmed to compute an indication of variation of reconstructed electrophysiological signals in respective sets of reconstructed signals. For example, the variation calculator 606 can compute the variation in reconstructed signal magnitude, such as representing a difference between signal values, such as signal amplitude (e.g., a difference between average amplitude, maximum amplitude or dominant frequencies) over a time interval.

In another example, the variation calculator 606 can be programmed to detect changes in electrophysiological signals that have been reconstructed on to the surface of interested based on a comparison one or more respective sets of the reconstructed electrophysiological signals with respect to baseline data. For example, the variation calculator 606 can determine which subsets of channels exhibit greater relative changes compared to the baseline data. The baseline data can be reconstructed electrophysiological signals from a time interval known to represent a normal cardiac rhythm for the patient. In another example, the baseline data can represent reconstructed electrophysiological signals that the reconstruction function 608 reconstructed based on a full (or nearly full) set of channels.

A signal emphasizer 610 can be programmed to emphasize (e.g., by weighting) a selected set of measurement channels, corresponding to electrophysiological signals measured by respective electrodes on the body surface, based on the reconstructed signal analysis 602. Signal emphasizer 610 can emphasize the signals by increasing the weight of the channels exhibiting the greater relative changes and/or and removing or decreasing the weight of channels exhibiting smaller changes relative to the baseline, such as described herein. For example, the signal emphasizer 610 can be programmed to selectively weight channels to increase the influence of those subsets of channels determined (e.g., by analysis and modifier function 602) to contribute to greater relative changes compared to the baseline data. Alternatively, or additionally, the function can decrease the weight of or remove respective channels exhibiting the smaller changes compared to the baseline data. As an example, signal emphasizer 610 can be programmed to increase or decrease weights applied to emphasize (or deemphasize) respective body surface channels exhibiting an amplitude change in percentage (e.g., 5% amplitude change compared to a baseline amplitude) or an absolute voltage change (e.g., 0.02 mV in atria and 0.1 mV in ventricle) after intervention when assessing the intervention's impact. As described herein, the mapping system 600 can include an output generator 618 is programmed to provide the output data 620 to visualize a graphical map or other information on a display (e.g., display 108) based on the reconstructed electrograms generated for one or more time intervals.

FIGS. 10A, 10B and 10C, 10D and 10E depict examples electrophysiological signals showing a comparison of signal waveforms, a set of patch electrodes and graphical maps provided for a full set of body surface electrodes and the set of patch electrodes.

For example, FIG. 10A shows a set of preprocessed signal waveforms 700, 702 and 704. The waveforms 700 represent signal channels measured by 248 electrodes distributed across a patient's thorax. The set of signal waveforms 702 includes an interval of measured signals for 197 of the 248 channels that have been determined to be good channels (e.g., by non-invasive signal analysis function 54). The set of signal waveforms 704 represents the same interval of measured signals for 13 channels of the measured by a set of patch electrodes, namely a patch having respective electrodes adapted to detect a PVC.

For example, FIGS. 10B and 10C are diagrams 710 and 712 of a front and back of a torso showing examples of patch electrodes 714 and 716, respectively, each having an arrangement of electrodes. The patch 714 includes six electrodes 706 configured in a circular arrangement, and the patch 716 includes seven patch electrodes 708 configured in a circular arrangement with an electrode near a center thereof. For example, the patches can each include a web (e.g., substrate) of pliant conformable material with an adhesive backing to attach to the sensing location shown in FIGS. 10 B and 10C. In other examples, the electrodes of the respective patches could be activated for sensing electrophysiological signal from a larger, full complement of electrodes.

FIGS. 10D and 10E show activation maps 720 and 722 for respective sets of sensed electrophysiological signals. For example, FIG. 10D shows the map 720 generated for the selected interval based on the signal waveforms shown at 702. The activation map 722 is generated (e.g., by output generator 144) for the respective electrophysiological signals sensed by patch electrodes 714 and 716. As a further example, a cross-correlation between the maps 720 and 722 can be computed to demonstrate a median cross-correlation of 0.98 and mean of 0.92 between the patch electrodes (e.g., 13 electrodes) and the full set of 197 electrodes.

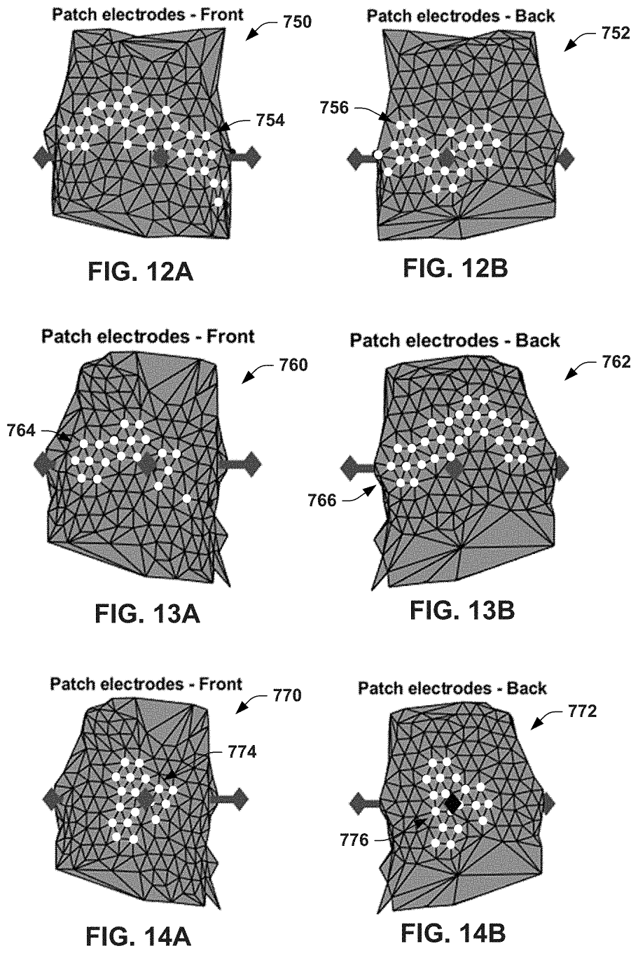

FIGS. 11A and 11B show potential maps 730 and 732, respectively, reconstructed for a cardiac envelope (e.g., the whole heart surface) based on electrophysiological signals measured by different electrode configurations and geometry data, such as described herein. The map 730 is generated based on electrophysiological signals (e.g., waveforms 700 or 702 shown in FIG. 10A) measured by a full set of electrodes and geometry data. The map 732 is generated based on electrophysiological signals measured by the patch electrodes 714 and 716 (e.g., derived from waveforms 704 shown in FIG. 10A) and geometry data.

The geometry data can include three-dimensional coordinates representing the entire cardiac envelope to where the EP signals are being reconstructed (e.g., a cardiac mesh). The geometry data can also include a three-dimensional representation for the torso (e.g., a torso surface mesh), including electrode locations used for measuring signals used to generate the maps 730 and 732. For the map 732 generated based on measurements from the patch electrode, the geometry data can represent a torso mesh including patch electrode locations and locations on the mesh without patch electrodes for measuring the signals. The potential maps 730 and 732 demonstrate that there is a high level of correlation between the full set of electrodes and the patch electrodes 714 and 716. FIG. 11C also includes a plot 734 of waveforms 736 and 738 selected from a respective point (e.g., the same anatomical spatial location—a node) on the cardiac envelope, shown at 740 and 742 in FIGS. 11A and 11B, respectively.

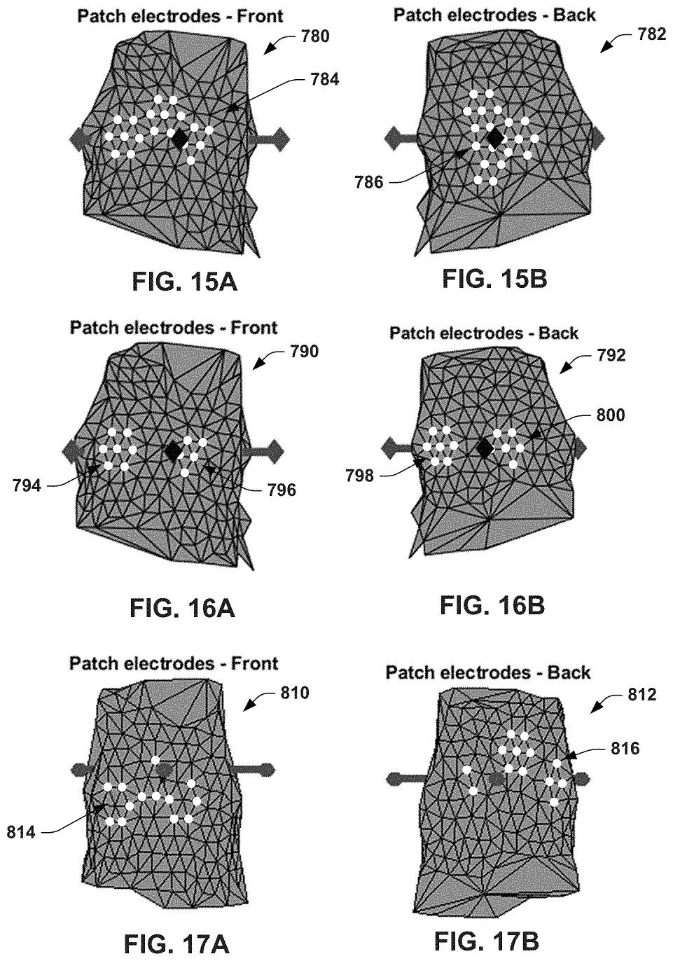

FIGS. 12A-17B are diagrams showing examples of different patch electrode configurations that can be implemented. The examples of FIGS. 12A-17B are not exhaustive and various other configurations can be realized, such as adapted to sense body surface electrophysiological signals associated with a localized arrhythmia (e.g., AF, PVC, AT, VT, or the like), for use during a particular procedure (e.g., CRT or Brugada syndrome) or otherwise having a specific geometry (e.g., a reduced set of electrodes adapted for simplified global or localized electrophysiological measurements).

For example, FIGS. 12A and 12B include front and back diagrams 750 and 752 showing an example of one or more patches in the form of a belt that can extend across the front and back portions of a patient's torso. For example, the belt includes a portion of patch electrodes 754 arranged and configured on a front portion of the torso and another portion of patch electrodes 756 arranged and configured on a back portion of the torso. The patch electrodes 754 and 756 are shown at locations adapted to measure electrophysiological signals indicating a PVC.

FIGS. 13A and 13B include front and back diagrams 760 and 762 showing another example of one or more patches in the form of a belt that can extend across the front and back portions of a patient's torso. For example, the belt includes a portion of patch electrodes 764 arranged and configured on a front portion of the torso and another portion of patch electrodes 766 arranged and configured on a back portion of the torso. The example patch electrode of FIGS. 13A-13B includes a greater number of electrodes on the back portion than the front portion.

FIGS. 14A and 14B include front and back diagrams 770 and 772 showing another example of multiple patches configured for sensing electrophysiological signals from the front and back portions of a patient's torso. For example, the patch electrode includes a first patch (or multiple patches) having a group of electrodes 774 arranged and configured on a central front portion of the torso and another group of electrodes 776 arranged and configured on a central back portion of the torso.

FIGS. 15A and 15B include front and back diagrams 780 and 782 showing another example of one or more patches configured for sensing electrophysiological signals from the front and back portions of a patient's torso. For example, a portion of patch electrodes 784 are arranged and configured in the form of a belt on a front portion of the torso and another portion of patch electrodes 786 arranged and configured as a central grouping of electrodes on the back portion of the torso.

FIGS. 16A and 16B include front and back diagrams 790 and 792 showing another example of patch electrodes configured for sensing electrophysiological signals from the front and back portions of a patient's torso. In the example of FIGS. 16A and 16B, patch electrodes 794 and 796 include respective groups of electrodes arranged and configured on a front portion of the torso. The patch electrodes 794 and 796 can be mounted to the same substrate, such as to maintain a desired spatial arrangement between the patch electrodes 794 and 796, or separate patch substrates could be used. Another set of patch electrodes 798 and 800 include respective groups of electrodes arranged and configured on a back portion of the torso. As with the front patch electrodes, the patch electrodes 798 and 800 can be mounted to the same substrate, such as to maintain a desired spatial arrangement between the patch electrodes, or separate patch substrates could be used.

FIGS. 17A and 17B include front and back diagrams 810 and 812 showing another example of one or more patches configured for sensing electrophysiological signals from the front and back portions of a patient's torso. As shown FIG. 17A, a portion of patch electrodes 814 are arranged and configured in the form semi-circular ends connected by a line of electrodes on a front portion of the torso. FIG. 17B show a portion of patch electrodes 816 arranged and configured on the back portion of the torso, including a central grouping and two side groupings lateral to the central grouping of electrodes. For example, the patch electrodes 814 and 816 can be adapted to measure electrophysiological signals during a focal atrial tachycardia (AT) for the right inferior pulmonary vein (RIPV). By using such an arrangement of electrodes, the contribution of other signals, which can dilute or otherwise obfuscate the components of the AT, can be reduced. In the example of FIGS. 17A and 17B, the patch electrodes include 27 sensors are arranged and configured to measure a set of electrograms that emphasize signal components that describe the AT condition measured from the body surface.

FIGS. 12A-17B thus show some examples of different patch electrodes, which can be implemented to sense a given arrhythmia condition and/or signals at one or more regions of interest, each of which can be specified in response to a user input or be derived automatically based on analysis of electrophysiological signals measured for one or more time intervals. In other examples, physical patches containing patch electrodes arranged in configured in a given manner can be implemented for sensing electrophysiological signal spatially and temporally relevant to sensing requirements.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system comprising:
an arrangement of body surface electrodes on one or more patches adapted to be placed an outer surface of a patient's body;
a computing apparatus including non-transitory memory to store data and instructions executable by a processor thereof, the data including:
anatomical geometry data representing anatomy of the patient, which includes a at least a portion of a heart and the outer surface of the patient's body, in three-dimensional space;
electrode geometry data representing locations of respective electrodes in three-dimensional space, the electrode geometry data derived from at least one of (i) electrical signals measured by at least some of the electrodes, or (ii) a template describing the locations of respective electrodes as applied to the patient; and
electrical data representing electrophysiological signals measured by the electrodes;
the instructions programmed to at least:
register the anatomical geometry data and the electrode geometry data to provide co-registered geometry data representing the anatomy of the patient and the locations of the electrodes in a common three-dimensional space; and
reconstruct electrophysiological signals on a cardiac envelope of the heart based on the co-registered geometry data and the electrical data.

2. The system of claim 1, the instructions further programmed to derive the anatomical geometry data from at least one of (i) imaging data acquired by a medical imaging modality, or (ii) a user-specific template describing the anatomy of the patient.

3. The system of claim 1, further comprising a probe adapted to be inserted in the patient's body, the probe comprising an electrode configured to provide an applied electrical signal, wherein the instructions further include a dipole model cost function having parameters representing a dipole location and moment corresponding to the applied electrical signal, the instructions applying a boundary condition on the dipole model cost function to determine a location of at least some of the electrodes on the outer surface relative to a known location of the probe and to generate the electrode geometry data based on the determined location of the electrodes.

4. The system of claim 1, further comprising a probe adapted to be inserted in the patient's body, the probe comprising an electrode configured to measure an intrabody electrophysiological signal at a known location within the patient's body, wherein the instructions are further programmed to compare a morphology of the measured intrabody electrophysiological signal with a morphology of the respective signals measured by the electrodes on the patient's body to generate the electrode geometry data based on the comparison.

5. The system of claim 4, wherein the instructions are further programmed to:
compute a forward calculation to determine a representation of the measured intrabody electrophysiological signal on the outer surface of the patient's body;
calculate angles from a known probe location within the patient's body to respective electrodes; and
determine the location of the respective electrodes with respect to the outer surface based on the forward calculation and the calculated angles.

6. The system of claim 1, wherein the instructions and/or data further comprise a look-up table that provides an estimate of the anatomical geometry data in response to input data representing physical parameters of the patient.

7. The system of claim 1, wherein the instructions are further programmed to:
select a proper subset of the electrodes;
calculate an inverse solution to reconstruct electrophysiological signals on the cardiac envelope based on the selected subset of electrodes; and
repeat the selecting and calculating for a plurality of subsets of electrodes to provide respective sets of reconstructed electrophysiological signals.

8. The system of claim 7, wherein the instructions are further programmed to combine together at least some of the respective sets of reconstructed electrophysiological signals into an aggregate set to seek convergence of the solution.

9. The system of claim 7, wherein the instructions are further programmed to:
compare the reconstructed electrophysiological signals on the cardiac envelope relative to baseline data to detect changes; and
emphasize a set of measurement channels, corresponding to electrophysiological signals measured non-invasively by respective electrodes, which exhibit greater relative changes responsive to the detected changes.

10. The system of claim 9, wherein the respective measurement channels are emphasized by at least one of increasing the weight of the respective measurement channels that exhibit the greater relative changes and removing or decreasing the weight of channels exhibiting smaller changes relative to the baseline data.

11. The system of claim 1, wherein the instructions are further programmed to:
emphasize electrophysiological signals for a subset of measurement channels, corresponding to electrophysiological signals measured non-invasively by respective electrodes, so the electrical data represents the emphasized electrophysiological signals for the subset of measurement channels together with other electrophysiological signals for other measurement channels, wherein the electrophysiological signals are reconstructed on the cardiac envelope of the heart based on the co-registered geometry data and the electrical data including the emphasized electrophysiological signals.

12. The system of claim 11, wherein the instructions are further programmed to:
compare respective electrophysiological signals measured by the electrodes on the outer surface, relative to baseline data representing baseline electrophysiological signals measured by the electrodes on the outer surface; and
identify the subset of measurement channels based on the comparison indicating greater relative changes for the electrodes that provide the subset of measurement channels.

13. The system of any of claim 9, wherein the baseline data represents electrophysiological signals prior to an intervention and the relative changes represent electrophysiological signals after the intervention.

14. The system of claim 11, wherein the subset of measurement channels are emphasized by at least one increasing the weight of the measurement channels exhibiting the greater relative changes and removing or decreasing the weight of measurement channels exhibiting smaller changes relative to a baseline.

15. The system of claim 11, wherein the instructions are further programmed to select the subset of measurement channels based on a spatial analysis of the electrode geometry data and/or a region of interest of the heart.

16. The system of claim 15, wherein the instructions are further programmed to:
compare electrophysiological signals measured by electrodes in each of a plurality of respective spatial neighborhoods of the electrodes; and
emphasize electrophysiological signals for electrodes in one or more of the respective spatial neighborhoods of the electrodes based on the comparison, the subset of measurement channels comprising the electrodes in the one or more of the respective spatial neighborhoods.

17. The system of claim 16, wherein the instructions are further programmed to:
determine a variation of the electrophysiological signals for each of the respective spatial neighborhoods; and
emphasize electrophysiological signals measured for electrodes in the respective spatial neighborhoods by an amount that is based on the variation of the electrophysiological signals determined among the respective spatial neighborhoods.

18. The system of claim 15, wherein the region of interest of the heart is based on position data representative of a location where a cardiac intervention is performed on the heart.

19. The system of claim 1, wherein at least one of a number or arrangement of the electrodes on the one or more patches are adapted to measure the electrophysiological signals for a given type of arrhythmia.

20. One or more non-transitory computer-readable media having instructions, which when executed by a processor, perform a method comprising:
emphasizing electrophysiological signals for a subset of measurement channels, corresponding to electrophysiological signals measured non-invasively by respective electrodes;
storing electrical data to represent the emphasized electrophysiological signals for the subset of measurement channels together with other electrophysiological signals for other measurement channels measured non-invasively by other electrodes; and
reconstructing electrophysiological signals on a cardiac envelope of a heart based on co-registered geometry data and the electrical data including the emphasized electrophysiological signals, the co-registered geometry data representing the anatomy of the patient and spatial locations of the respective electrodes in a common three-dimensional space, and the electrical data representing electrophysiological signals measured non-invasively by electrodes corresponding to respective measurement channels.

21. The media of claim 20, wherein the method to be performed by the instructions further comprises:
comparing respective electrophysiological signals measured by the electrodes on an outer surface of the patient's body relative to baseline data representing baseline electrophysiological signals measured by the electrodes on the outer surface; and
identifying the subset of measurement channels based on the comparison.

22. The media of claim 21, wherein the baseline data represents electrophysiological signals prior to an intervention and the relative changes represent electrophysiological signals after the intervention.

23. The media of claim 20, wherein the subset of measurement channels are emphasized by at least one increasing the weight of the measurement channels exhibiting the greater relative changes and removing or decreasing the weight of measurement channels exhibiting smaller changes relative to a baseline.

24. The media of claim 20, the method to be performed by the instructions further comprises selecting the subset of measurement channels based on a spatial analysis of the spatial locations of the respective electrodes and/or a region of interest of the heart.

25. The media of claim 24, wherein the method to be performed by the instructions further comprises:
comparing electrophysiological signals measured by electrodes in each of a plurality of respective spatial neighborhoods of the electrodes; and
emphasizing electrophysiological signals for electrodes in one or more of the respective spatial neighborhoods of the electrodes based on the comparison, the subset of measurement channels comprising the electrodes in the one or more of the respective spatial neighborhoods.

26. The media of claim 25, wherein the method to be performed by the instructions further comprises:
determining a variation of the electrophysiological signals for each of the respective spatial neighborhoods; and
emphasizing electrophysiological signals measured for electrodes in the respective spatial neighborhoods by an amount that is based on the variation of the electrophysiological signals determined among the respective spatial neighborhoods.

27. One or more non-transitory computer-readable media having instructions, which when executed by a processor, perform a method comprising:
selecting a proper subset of a plurality of measurement channels, the measurement channels corresponding to arrangement of electrodes on one or more patches adapted to measure electrophysiological signals on an outer surface of a patient's body;
storing electrical data to represent the electrophysiological signals measured for the selected subset of measurement channels;
reconstructing electrophysiological signals on a cardiac envelope of a heart based on co-registered geometry data and the electrical data for the selected subset of measurement channels, the co-registered geometry data representing the anatomy of the patient and spatial locations of the electrodes in a common three-dimensional space; and
repeating the selecting, storing and reconstructing for a plurality of subsets of electrodes to provide respective sets of reconstructed electrophysiological signals.

28. The media of claim 27, wherein the method to be performed by the instructions further comprises combining together at least some of the respective sets of reconstructed electrophysiological signals into an aggregate set.

29. The media of claim 27, wherein the method to be performed by the instructions further comprises:
compare the reconstructed electrophysiological signals on the cardiac envelope relative to baseline data to detect changes; and
emphasize a set of measurement channels, corresponding to electrophysiological signals measured non-invasively by respective electrodes, which exhibit greater relative changes responsive to the detected changes.

30. The media of claim 29, wherein the respective measurement channels are emphasized by at least one of increasing the weight of the respective measurement channels that exhibit the greater relative changes and removing or decreasing the weight of channels exhibiting smaller changes relative to the baseline data.
